SEQUENCE LISTING (40 sequences)

SEQ ID NO: 1 - DNA, 1341 bp, Synechocystis sp. strain PCC6803
  CDS (1)..(1341)
atgactaacccttttggaagacgtaaatttttgctgtatggatcagcaaccctaggcgccagtctattgctcaaagcctgtggcggcggcacggaacctaccaccgaacccactgctgaaccgactgagtcccccaccaccggtactgctcccaccggggaaccgattaaagttggtttgctccactccctcagtggcaccatggccatcagtgaaaccaccgtggtggaagcggcggaactggcgatcgaagagatcaatgcggccggtggagttttgggtagacccattgaagccatcaaagaagatggagcttccgattggccgacttttgcggaaaaagcggctaagttaattgaccaagataaggtacccgtagtctttggttgttggacttccgccagccggaaagcggtactgccggtatttgaagccaaaaatcatatgctttggtacccagtacagtacgaaggtcaggaatgttccaaaaacattttctacaccggtgccgcccccaaccaacaaattgaaccggcggtggattggttgctggaaaataaaggcaataagttcttcctggtgggttccgattacgttttcccccgcactgctaacaccatcattaaagagcagttgaaagccaaaggtggcgaaacccttggggaagattacctacccctgggtaacaccgaagttacccctatcatcaccaaaatccgggaagccctgcccgatggcggcgtaattttcaacaccctgaatggggacagtaatgttgccttcttcaagcagatccaagccgctggtttgacccccgataaatatccggtcatgtccgtgagtgtggcggaagaggaagtacgtcaaattggtaaggagtatctgctcggccagtttgcttcttggaactatttccagagtgtggatacccctgccaaccaaaaatttgtggcagcttttaaggctaaatacggtgaagaccgggtgaccaacgaccccatggaagcagcttatatttccgtttacctctggaaggcggcggtggaagcggctggagatgtgggtgaaactcccgaaggcttagaaaaagtccgggcggcggcgattggtaaaacctttgacgctccggaaggcatggtgaccatgcaacccaaccaccacatttccaaaactgtccgcattggggaagtcaatgacgaaggtcagttcaccattgtttggtccagtgatggccccgtggaccccattccctggaaccagttcgtaccggaaaccaaaggtttcacctgcgattggacccgcacagatgtggaaaatcctggtaagttcaaggccagctaa

SEQ ID NO: 2 - protein, 446 aa, Synechocystis sp. strain PCC6803
MTNPFGRRKFLLYGSATLGASLLLKACGGGTEPTTEPTAEPTESPTTGTAPTGEPIKVGLLHSLSGTMAISETTVVEAAELAIEEINAAGGVLGRPIEAIKEDGASDWPTFAEKAAKLIDQDKVPVVFGCWTSASRKAVLPVFEAKNHMLWYPVQYEGQECSKNIFYTGAAPNQQIEPAVDWLLENKGNKFFLVGSDYVFPRTANTIIKEQLKAKGGETLGEDYLPLGNTEVTPIITKIREALPDGGVIFNTLNGDSNVAFFKQIQAAGLTPDKYPVMSVSVAEEEVRQIGKEYLLGQFASWNYFQSVDTPANQKFVAAFKAKYGEDRVTNDPMEAAYISVYLWKAAVEAAGDVGETPEGLEKVRAAAIGKTFDAPEGMVTMQPNHHISKTVRIGEVNDEGQFTIVWSSDGPVDPIPWNQFVPETKGFTCDWTRTDVENPGKFKAS

SEQ ID NO: 3 - DNA, 417 bp, Synechocystis sp. strain PCC6803
  CDS (1)..(417)
gtgttcatgcaaactaagcacatagctcaggcaacagtgaaagtactgcaaagttacctcacctaccaagccgttctcaggatccagagtgaactcggggaaaccaaccctccccaggccatttggttaaaccagtatttagccagtcacagtattcaaaatggagaaacgtttttgacggaactcctggatgaaaataaagaactggtactcaggatcctggcggtaagggaagacattgccgaatcagtgttagattttttgcccggtatgacccggaatagcttagcggaatctaacatcgcccaccgccgccatttgcttgaacgtctgacccgtaccgtagccgaagtcgataatttcccttcggaaacctccaacggagaatcaaacaacaacgattctcccccgtcctaa

SEQ ID NO: 4 - protein, 138 aa, Synechocystis sp. strain PCC6803
VFMQTKHIAQATVKVLQSYLTYQAVLRIQSELGETNPPQAIWLNQYLASHSIQNGETFLTELLDENKELVLRILAVREDIAESVLDFLPGMTRNSLAESNIAHRRHLLERLTRTVAEVDNFPSETSNGESNNNDSPPS

SEQ ID NO: 5 - DNA, 786 bp, Synechocystis sp. strain PCC6803
  CDS (1)..(786)
atgcatttagttgatacccatgtccacattaactttgatgtttttgcggcggatttagaccagttacagcatcgctggcggcaagctggggtggtgcaactggttcattcctgcgttaagccccaggagtttgatcaaatacagtctctggcggaccgttttcctgaactatttttcgccgtgggactccatcctttggatgccgaagattggcaagacaatactgctgggcaaatccttgcctatgccaaggcggatgaccgggtggtagccattggtgaaatgggtttggattttttcaaagccgataaccgtgaccatcaaattgaggttttccgggcccagttggcgatcgccagggaattaaacaagccagtgattatccattgtcgggatgccgcccagaccatgcgccaggtattgactgatttccaagcagaatcggggcccgtggctggggtaatgcactgttggggtggcactcctgaagaaacccaatggtttttggacctggggttttacatcagttttagcggcacagttaccttcaaaaaagctgaagggatccaagccagtgcccagatggtcccccccgatcgcctgttggtggaaaccgattgtccctttttggcgccagtgccccaacggggtaaacgcaatgaaccagcctttgtccgccatgtggccgaggcgatcgctgccctgcgccatgtccccctagaaacccttgcccaacaaaccactactaatgcccgcaacctttttaaactaccggtgcctgcctaa

SEQ ID NO: 6 - protein, 261 aa, Synechocystis sp. strain PCC6803
MHLVDTHVHINFDVFAADLDQLQHRWRQAGVVQLVHSCVKPQEFDQIQSLADRFPELFFAVGLHPLDAEDWQDNTAGQILAYAKADDRVVAIGEMGLDFFKADNRDHQIEVFRAQLAIARELNKPVIIHCRDAAQTMRQVLTDFQAESGPVAGVMHCWGGTPEETQWFLDLGFYISFSGTVTFKKAEGIQASAQMVPPDRLLVETDCPFLAPVPQRGKRNEPAFVRHVAEAIAALRHVPLETLAQQTTTNARNLFKLPVPA

SEQ ID NO: 7 - DNA, 213 bp, Synechocystis sp. strain PCC6803
  CDS (1)..(213)
atgactagccgcggatttcgcctcgaccaagacaaccgtctcaacaacttcgccattgaaccccctgtgtacgttgacagcagtgttcaagccggttggactgaatacgccgaaaaaatgaatggtcgttttgccatgattggctttgtttctctcttggcaatggaagtaattactggccacggcattgtgggttggttgctctctctctaa

SEQ ID NO: 8 - protein, 70 aa, Synechocystis sp. strain PCC6803
MTSRGFRLDQDNRLNNFAIEPPVYVDSSVQAGWTEYAEKMNGRFAMIGFVSLLAMEVITGHGIVGWLLSL

SEQ ID NO: 9 - DNA, 312 bp, Synechocystis sp. strain PCC6803
  CDS (1)..(312)
atgaactaccaaaggactgcccttggcaccgtgaaaatcgaacaaataagaggtaaaactatgaacgccgacactgatatttatcaaaacaaagatctatttgcccccgttgtcttccgcaaagacttcaaccaatttgcccccatcaacgggaaccaagcctggtctttatttttcaccgccgggcaagaagataagcaactgggcaacagccctgaattcggtcgctttttcaccaatactctcttcgccattggggctgccactttcatctggggttacttcttcagccgttgggctgactttctctaa

SEQ ID NO: 10 - protein, 103 aa, Synechocystis sp. strain PCC6803
MNYQRTALGTVKIEQIRGKTMNADTDIYQNKDLFAPVVFRKDFNQFAPINGNQAWSLFFTAGQEDKQLGNSPEFGRFFTNTLFAIGAATFIWGYFFSRWADFL

SEQ ID NO: 11 - DNA, 1140 bp, Synechocystis sp. strain PCC6803
  CDS (1)..(1140)
atgttaagcctcagtttaggggggcagtttatgaacaacaatatccgcgtcggcagtctgtttggcattcctttttacgtcaacccatcctggtttttaattttaggattggtgaccctgagctatggccaagacttagcccgctttccccaactttccggtggcacaccctggattttggggttaattacagctttactcctctttgcttccgttgtcgcccacgagttgggccatagtttggttgccttagcccagggcattgaagttaaatccatcactctgtttttgttcggtggtctagcgagtttagaaaaggaatccaacactccctggcaagcttttgcggtggcgatcgccgggccggcggtgagtttagtgctctttttgggtttaaccatagttggtacccaaatccccctacctgtgccggggcaggccatcattggtttattgggcatgatcaacctcgccctggcattgtttaacctcattcctggtttacctttggacggcggcaatgtgctcaaatccattgtgtggcaaatcacgggcaatcaaaacaaaggtattctcattgctagtcgggtgggccagggtttcggttggttggcgatcgccattggtagcttaggtattttaaatattctgcccatcggtagcttctggaccattttgatcggttggttcctgttacaaaatgctggttcctccgcccgcaacgcccaggtcaaagagcaaatggaagcctttactgctgaagatgcggttattcccaacagccccattattcctgccgggttaaatattcgggaatttgctaacgattatgtgattggtaaaaccccctggcgacggttcttggttattggtgccgacaatcaactgttaggtgtacttgctacggaagacatcaaacacgtccccacttccgattggccccaggtcacagtggatagcttgatgcagtatccccaacagatggtcaccgttaacgccaatcaatctttgtttgaagtggcccagttgttagatcaacagaaactgtcggaacttttggtggtgcaaccttcgggagaagtggtgggattattggaaaaagcttccatcatcaaatgtctgcaaacctccgccgcctag

SEQ ID NO: 12 - protein, 379 aa, Synechocystis sp. strain PCC6803
MLSLSLGGQFMNNNIRVGSLFGIPFYVNPSWFLILGLVTLSYGQDLARFPQLSGGTPWILGLITALLLFASVVAHELGHSLVALAQGIEVKSITLFLFGGLASLEKESNTPWQAFAVAIAGPAVSLVLFLGLTIVGTQIPLPVPGQAIIGLLGMINLALALFNLIPGLPLDGGNVLKSIVWQITGNQNKGILIASRVGQGFGWLAIAIGSLGILNILPIGSFWTILIGWFLLQNAGSSARNAQVKEQMEAFTAEDAVIPNSPIIPAGLNIREFANDYVIGKTPWRRFLVIGADNQLLGVLATEDIKHVPTSDWPQVTVDSLMQYPQQMVTVNANQSLFEVAQLLDQQKLSELLVVQPSGEVVGLLEKASIIKCLQTSAA

SEQ ID NO: 13 - DNA, 441 bp, Synechocystis sp. strain PCC6803
  CDS (1)..(441)
atgtctctcattctttacaatcccctgcgggaaatggataatttccagcagcagatgaaccaactgtttgaagaagtttttgtccctacggaccgccacggcgatcgccaagggtttaatcctaaagcagaactaactgaaactgaagaagcctatgtgctcaaactagaattacctggcatggaccccgataatttggacatccaagccgccagggatgcggtgaccgtcagcggcgatcgccaggatacccatagcaccgaaaaagatggggtgcggcgcacagagttccgctatggcagtttccgccgggttattcctgtacctggagcaatccaaaacacagaagttaaagctaattacgatgccggtatcctaactttgactttgcccaaagtagaggaagccaaaaataaagtggtgaaagttcagctttcctaa

SEQ ID NO: 14 - protein, 146 aa, Synechocystis sp. strain PCC6803
MSLILYNPLREMDNFQQQMNQLFEEVFVPTDRHGDRQGFNPKAELTETEEAYVLKLELPGMDPDNLDIQAARDAVTVSGDRQDTHSTEKDGVRRTEFRYGSFRRVIPVPGAIQNTEVKANYDAGILTLTLPKVEEAKNKVVKVQLS

SEQ ID NO: 15 - DNA, 702 bp, Synechocystis sp. strain PCC6803
  CDS (1)..(702)
atggcagaagaaattctcagaaacccagccatgacagccctgaccctcgaacaaattgccagccaactcgacagccccaattcccgcgatcgcctgattgccctagcttccctgagaccctattccagtgaggaggcggtgcccctgattaaaaaagttttagatgacgatactttacaggtgcgttccatggcggtgtttgccctgggcattaagcaaaccgaggaatgctatcccattctggttaagctgttggaaaccgatggagactatggcatccgggccgatgccgcgggggccctgggttatctagaagacgaacgggctttccatcccctctgccgggctttttacgaagatacggaatggctggtgcggttcagtgcggcggtggccctgggcaatttaaaagatattcgggctcaaacggtcttgctggaagcactgaaaagtgacgaagcagtggtacaacaagcggcgatcgcggccctgggggaaattggtgccgtggatgcagtagatgcgattttggcctttgcatcccatgaggactggttaattcgccaaagattagtggaggccctgggaaatttgccctgcgaccagagtcgttctgctttgactttcatggtcaaggatgagcacccccaggtgtcccaggcggcccagttgtccttgcaaaaattagacctgcttagctag

SEQ ID NO: 16 - protein, 233 aa, Synechocystis sp. strain PCC6803
MAEEILRNPAMTALTLEQIASQLDSPNSRDRLIALASLRPYSSEEAVPLIKKVLDDDTLQVRSMAVFALGIKQTEECYPILVKLLETDGDYGIRADAAGALGYLEDERAFHPLCRAFYEDTEWLVRFSAAVALGNLKDIRAQTVLLEALKSDEAVVQQAAIAALGEIGAVDAVDAILAFASHEDWLIRQRLVEALGNLPCDQSRSALTFMVKDEHPQVSQAAQLSLQKLDLLS

SEQ ID NO: 17 - DNA, 543 bp, Synechocystis sp. strain PCC6803
  CDS (1)..(543)
atgaaaaccgctgctagaattgttgcttttaccgctctgactggatttgccctggggatgcccaccgttgccatggcggaaatggaaaccaccgaaaaatctgccgtagttagtcaagccgccacggacagcgccatgactattgtggaagtcgccgcaggcaatgaaactttcagtaccctcgttgcagcagtcaaagcggctgatttagtggaagctttatccgctgaaggcccctttaccgtttttgcccccaccaatgatgcctttgccgctctgcccgctggtacggtggaaagtctgttgttgcccgaaaacaaagataaattggtgaaaattttgacctaccacgtcgttcctggcaaaatcaccgccgcccaggttcaatccggtgaagtggcatccctagctggggaagccctcaccttcaaagtcaaagatggcaaagtgaaagttaacaaagccactgtcatttccgccgatgtggatgccagcaacggtgtaatccatgtcattgaccaagtaattctgcctcctatgtaa

SEQ ID NO: 18 - protein, 180 aa, Synechocystis sp. strain PCC6803
MKTAARIVAFTALTGFALGMPTVAMAEMETTEKSAVVSQAATDSAMTIVEVAAGNETFSTLVAAVKAADLVEALSAEGPFTVFAPTNDAFAALPAGTVESLLLPENKDKLVKILTYHVVPGKITAAQVQSGEVASLAGEALTFKVKDGKVKVNKATVISADVDASNGVIHVIDQVILPPM

SEQ ID NO: 19 - DNA, 957 bp, Synechocystis sp. strain PCC6803
  CDS (1)..(957)
atgactgccagaaccagccccgattccgtccgtgcctatctcagagaaattggtcgtgtgcccctgctcacccatgaggaagagattgtttatgctaagcaaatccaacaggttgttagcctcaacgaaatcaagaagtctttggccgaaggcaaggatggcgagccggtttcccccagcgagtgggctaaggcggccgatttgtccattcgagaattagaaaaagccatcaaggaaggggaacgggccaagcgcaaaatggtggaggctaacctccggctggtggtatctgtcgccaaaaaatatctcaagcgtaatctagacctacttgacctcatccaagagggcaccattggtatgcaacggggggtagagaagtttgaccccaccaagggttatcggttttccacctatgcctattggtggatccgccaggccatcaccagggcgatcgccgaaaagagccgcaccatccgtttaccaatccacattacggaaaagttaaacaaaattaaaaaagcccaaagacaactttcccaggaaaagggtcgggccgcttccattgcggaattggcggaacatctagaattaactcccaagcaagtgcgggaatatttggagcgctctcgccatcccctttccttggatttacgggtgggggacaaccaagatactgagttaggggatttgttggaagacgacggtcctttaccagaggattttgccacctatgcctccctacagttggatctcgatagcctgatggcggaattaacgccccaacaacgggaagttctcattctccgctttggcctcaatgatggccaacccctaaccttggcgagcattggctccatgctcagcatcagtcgggaacgggtgcggcagattgagcgggaagccctaaataaattacgcaaacgcaagtccatgatccaggaatatttagctagctaa

SEQ ID NO: 20 - protein, 318 aa, Synechocystis sp. strain PCC6803
MTARTSPDSVRAYLREIGRVPLLTHEEEIVYAKQIQQVVSLNEIKKSLAEGKDGEPVSPSEWAKAADLSIRELEKAIKEGERAKRKMVEANLRLVVSVAKKYLKRNLDLLDLIQEGTIGMQRGVEKFDPTKGYRFSTYAYWWIRQAITRAIAEKSRTIRLPIHITEKLNKIKKAQRQLSQEKGRAASIAELAEHLELTPKQVREYLERSRHPLSLDLRVGDNQDTELGDLLEDDGPLPEDFATYASLQLDLDSLMAELTPQQREVLILRFGLNDGQPLTLASIGSMLSISRERVRQIEREALNKLRKRKSMIQEYLAS

SEQ ID NO: 21 - DNA, 213 bp, Synechocystis sp. strain PCC6803
  CDS (1)..(213)
atgggcgcaatactctgttacatttatttacatagacaaccctcccagctcgtaattacattcttaaccatgaacaacgaaaactctaaatttggattcactgctttcgccgaaaactggaatggtcgcttggccatgatcggtttttcctctgccctgatcctcgagcttgtctctgggcaaggtgtacttcacttcttcggcattctgtaa

SEQ ID NO: 22 - protein, 70 aa, Synechocystis sp. strain PCC6803
MGAILCYIYLHRQPSQLVITFLTMNNENSKFGFTAFAENWNGRLAMIGFSSALILELVSGQGVLHFFGIL

SEQ ID NO: 23 - DNA, 213 bp, Synechocystis sp. strain PCC6803
  CDS (1)..(213)
atgaccacccgtggcttccgcttggatcaggacaaccgtctcaacaactttgccatcgaaccagaggtttacgtcgactcttccgtacaagcgggttggactaaatacgccgaaaaaatgaatggtcgtttcgccatgattggttttgcctccctccttattatggaagtggtcacagggcacggcgtcattggttggttaaatagcctgtag

SEQ ID NO: 24 - protein, 70 aa, Synechocystis sp. strain PCC6803
MTTRGFRLDQDNRLNNFAIEPEVYVDSSVQAGWTKYAEKMNGRFAMIGFASLLIMEVVTGHGVIGWLNSL

SEQ ID NO: 25 - DNA, 309 bp, Synechocystis sp. strain PCC6803
  CDS (1)..(309)
atgaaattttccctcgagtctctctatggttggtaccgtcaaatgctgaaccatccccggtaccgttggtggattgtcctcggctccttggtgtatctcctcagtcccatcgattttctgcccgacgttttccccgtacttggttggattgacgatggtttaattgccactttgctggtatcggaaatttcccaaatggttctcactggcttaaaaaacaagacaaccaagcaggaaaaggatgccccccaggaaaccgtggtggtggatgtggtggatgtggtgggacaggacgtggcccacagttaa

SEQ ID NO: 26 - protein, 102 aa, Synechocystis sp. strain PCC6803
MKFSLESLYGWYRQMLNHPRYRWWIVLGSLVYLLSPIDFLPDVFPVLGWIDDGLIATLLVSEISQMVLTGLKNKTTKQEKDAPQETVVVDVVDVVGQDVAHS

SEQ ID NO: 27 - DNA, 351 bp, Synechocystis sp. strain PCC6803
  CDS (1)..(351)
gtgacccatgaaccccaacgtccccaaccgttattcgctggcaatgaagccccaggcaaagatagtttgtggacatacgttcaagaattaagccccgaaaccattgcccaattatctcgccccgattcccaggaagtgtttcaggtgatggagcgcaacattatcggtctgttgggaaatttacccccggagcactttggggtaaccatcagcactagccgggaaaatttgggccgtcttttagcctccgccatgatgagtggctattttcttcgcaacgccgagcaaaggttaggatttgaacaagcttttaaaagtagcagcaacagcaacgagaataccgaatactaa

SEQ ID NO: 28 - protein, 116 aa, Synechocystis sp. strain PCC6803
VTHEPQRPQPLFAGNEAPGKDSLWTYVQELSPETIAQLSRPDSQEVFQVMERNIIGLLGNLPPEHFGVTISTSRENLGRLLASAMMSGYFLRNAEQRLGFEQAFKSSSNSNENTEY

SEQ ID NO: 29 - DNA, 1851 bp, Synechocystis sp. strain PCC6803
  CDS (1)..(1851)
gtgagcaaaaataataaaaaatggcgtaacgcgggcctatatgccttgttgttaattgtcgttttagcgttggcatcggcctttttcgaccgaccgacccaaactagggaaaccctcagctacagcgattttgtcaatcgggtagaagccaatcagatcgaacgggtcaacctcagtgccgaccgcacccaagcccaagtacccaatcccagcggtggtcctccctacttagtcaatctgcccaacgaccccgacttgatcaatattctcacccaacacaacgtggatattgctgtccaaccccagagcgacgaaggtttctggttccgcatcgccagcaccctatttttgcccatcttgctcttggtgggaatttttttcctcttccgtcgggcccagagtggccctggttcccaagccatgaactttggtaaatccaaagcacgggtgcaaatggaaccccaaacccaagttaccttcggggacgtggccggtattgagcaagccaaactagaactcaccgaagtggtggacttcctgaaaaatgcagaccgcttcaccgaattgggagccaaaattcccaagggtgttttgttggtaggcccccccggaaccggtaaaaccctgttggccaaagccgtggctggggaagcgggtgtaccgttcttttccatctccggttcggaatttgtggaaatgtttgtcggtgttggtgcttctcgggtacgggatttgtttgagcaggctaaagccaatgctccctgtatcgtcttcatcgatgaaattgatgccgttggtcgtcaacggggcgctggccttggtggtggtaatgatgagcgggaacagaccctcaaccagttgctaacggaaatggacggttttgaaggcaacaccggcattattatcgtcgccgccactaaccgtcccgatgtattggattctgccttgatgcgtcccggtcgtttcgatcgccaagtggtagtagaccgtcctgattatgctggccgtcgagaaatcctcaatgtccatgcccggggtaaaaccctttcccaggatgtggatttggataaaattgcccgtcgtacccctggatttaccggtgctgacctgtccaacctgttgaacgaagccgctattttggctgcccgtcgcaacttgaccgaaatttccatggacgaagtcaacgacgccattgaccgggtgttggctggtcctgagaagaaaaatcgggtgatgagcgaaaaacgcaaaaccctagtggcttaccatgaagctggccacgccttggtgggtgctttgatgcctgattatgatccagtacaaaaaattagcattattccccgcggccgggccggtggtttaacctggttcacccccagtgaagaccgtatggaatccggtttatactcccgttcctatctgcaaaatcagatggccgttgccctgggaggccgtattgctgaggaaattattttcggcgaagaggaagtcaccaccggtgcttccaacgacctccaacaggtagcccgggtcgcccgccaaatggtaacccgtttcggcatgagcgatcgcctgggcccggtagctttgggtcgtcagggtggtggggtattccttggtcgggacattgcctctgaccgggacttttccgatgaaaccgctgcggcgatcgatgaggaagtaagtcaattggtagaccaagcctatcaacgggccaaacaggtcttggtggaaaaccgtggcattttagatcaactggcagaaatcttggtagaaaaggaaactgttgattctgaagagctgcaaactctcctggctaacaacaatgccaaattggcacttctagtttaa

SEQ ID NO: 30 - protein, 616 aa, Synechocystis sp. strain PCC6803
VSKNNKKWRNAGLYALLLIVVLALASAFFDRPTQTRETLSYSDFVNRVEANQIERVNLSADRTQAQVPNPSGGPPYLVNLPNDPDLINILTQHNVDIAVQPQSDEGFWFRIASTLFLPILLLVGIFFLFRRAQSGPGSQAMNFGKSKARVQMEPQTQVTFGDVAGIEQAKLELTEVVDFLKNADRFTELGAKIPKGVLLVGPPGTGKTLLAKAVAGEAGVPFFSISGSEFVEMFVGVGASRVRDLFEQAKANAPCIVFIDEIDAVGRQRGAGLGGGNDEREQTLNQLLTEMDGFEGNTGIIIVAATNRPDVLDSALMRPGRFDRQVVVDRPDYAGRREILNVHARGKTLSQDVDLDKIARRTPGFTGADLSNLLNEAAILAARRNLTEISMDEVNDAIDRVLAGPEKKNRVMSEKRKTLVAYHEAGHALVGALMPDYDPVQKISIIPRGRAGGLTWFTPSEDRMESGLYSRSYLQNQMAVALGGRIAEEIIFGEEEVTTGASNDLQQVARVARQMVTRFGMSDRLGPVALGRQGGGVFLGRDIASDRDFSDETAAAIDEEVSQLVDQAYQRAKQVLVENRGILDQLAEILVEKETVDSEELQTLLANNNAKLALLV

SEQ ID NO: 31 - DNA, 1596 bp, Synechocystis sp. strain PCC6803
  CDS (1)..(1596)
atgatcgatcgccttttgtacgttcgtcttccctgtaacccgattttccccattggggtgatttatctggcggaccatgtccataaatgttttccggcgaccgcccagcggattttcgatttaggcaccattcctcccctggatttcaaccgggcccttgatgaatgtattgatgaatttcagccgacaattttggttttttcctggcgggacattcaaatctatgctccggtggggggtaggggtggtaatcccctgcagaacgcgtttgagttttactacggaaaaaatcccttggtgaagctgaggggagccttaggtggtttgaaagttaccagtgcctattacggcgaattatggcgtaatttaagactaataaaccggggattgcggagggcaaagcgttattgcagtgatccccaaatcattgtcgggggcggagcagttagtgttttttacgaacagttaaaaaccaagttgccagcgggcaccattgtgtctgtgggagaaggggaaaccctgttagaaaaatatctacgggggcaaaccattgaagacgaacggtgttacatagtcggccgcagtcagccccggccccggttaatccatgaacagccctcccccatggtaaaaactgcctgtgattatgactacatcgagcaaatttggccggcctttgactattacctccaggaggatgatttttacctaggggtacaaactaagcggggttgtccccacaattgctgttactgcgtttacaccgtggtggaagggaaacaggtcagaattaatcccgccgccgaagtggtcaaggaaatgcggcaactttatgaccggggcattcgcaatttttggttcaccgatgctcaatttattccggctagggtttttatagatgatgtggtggaattgctggaggcgatcgccgcgtcgggcatggaggatatccattgggctgcctatatccgagctgacaatttaacccctcggttgtgtgaactgatggtacaaacggggatgaactactttgaaattggtatcaccagtggttcccaggaattggtacgcaaaatgcgcatgggttacaatctccgcaccgtgttacagaattgtcgggatctaaagggggcaggctttaatgatttggtttccgtcaattattccttcaatgttattgatgaaaccctagacaccatccgccaaaccattgcctaccatcgggagttagaagctatttttggggcagacaaagtagaaccagccattttcttcattgggctacagccccacacccatctggaaacctatgccctggacaaggaaattctcaaaccaggctatgaccccatgagcatgatgccctggaccgccaaaaaattactctggaatccagaacccctcggctcgtttttcggcgaagtttgcctccaggcttggcaacaaaatcccaatgatttcggtcgagaagtgatgaatattctcgagcaacggctgggcaaagctgatctcgaaacagccctccactcccccctgcccgacaaaaaaaaatttccccctaccatggcagagggaaaaaaactcagtcctatttag

SEQ ID NO: 32 - protein, 531 aa, Synechocystis sp. strain PCC6803
MIDRLLYVRLPCNPIFPIGVIYLADHVHKCFPATAQRIFDLGTIPPLDFNRALDECIDEFQPTILVFSWRDIQIYAPVGGRGGNPLQNAFEFYYGKNPLVKLRGALGGLKVTSAYYGELWRNLRLINRGLRRAKRYCSDPQIIVGGGAVSVFYEQLKTKLPAGTIVSVGEGETLLEKYLRGQTIEDERCYIVGRSQPRPRLIHEQPSPMVKTACDYDYIEQIWPAFDYYLQEDDFYLGVQTKRGCPHNCCYCVYTVVEGKQVRINPAAEVVKEMRQLYDRGIRNFWFTDAQFIPARVFIDDVVELLEAIAASGMEDIHWAAYIRADNLTPRLCELMVQTGMNYFEIGITSGSQELVRKMRMGYNLRTVLQNCRDLKGAGFNDLVSVNYSFNVIDETLDTIRQTIAYHRELEAIFGADKVEPAIFFIGLQPHTHLETYALDKEILKPGYDPMSMMPWTAKKLLWNPEPLGSFFGEVCLQAWQQNPNDFGREVMNILEQRLGKADLETALHSPLPDKKKFPPTMAEGKKLSPI

SEQ ID NO: 33 - DNA, 1038 bp, Synechocystis sp. strain PCC6803
  CDS (1)..(1038)
atggtaacagtgacagttattctgttgctcttcattaaggagtcattccgaatgcccaccgccaatctctcctcccctacctccccccccactttcaccgccgatatggtgaggtcctatctccatgaaattggtcgggtacccctgttaacccatgagcaagaaattatcctcggtaaacaagtccaacaaatgatggccctgctggagcacaagaaagccctggctgacagattgggccgagagccctccgacccggaatgggcggaagcggcggatttgtcggtgacgaaattacaccgctatctgggccaaggggaacgggccaaacggaaaatgattgaagctaacctccggttggtggtggcgatcgccaagaaatatcagaagcgcaatatggagtttttggatttgatccaagaaggtagcctgggtttagaacggggggtggaaaaattcgaccccaccaagggttataaattctccacctatgcctactggtggattcgccaagccatcacccgggcgatcgcccaacagggccggactatccgtttgcccattcatatcactgaaaagttaaacaaaatcaaaaaaacccaacgggaactttcccaacaattgggccgcagtgccacccccgccgaagtagccaaggctctggaaattgaccctagtcaaattcgcgagtacctcagtctgtcgcgccaacccatctccctcgatgtgcgggtgggggataatcaggacacagaattgtccgaactcttggaggacgaaggggtttcccccgatgcttacatcacccaggagtccatgcgtcaagacctgcaaaatttactggcggaattaacaccccagcaacaggctgtgctgaccatgcgttttggtcttaacgatggccaagagctatctttggctaaaatcggccagcatctcaacatcagccgggaaagggtccgccaattagaaaaccaagcccttgcgcaactgaagcgtcggcgggctaatatggcagagtatattatcgccagttag

SEQ ID NO: 34 - protein, 345 aa, Synechocystis sp. strain PCC6803
MVTVTVILLLFIKESFRMPTANLSSPTSPPTFTADMVRSYLHEIGRVPLLTHEQEIILGKQVQQMMALLEHKKALADRLGREPSDPEWAEAADLSVTKLHRYLGQGERAKRKMIEANLRLVVAIAKKYQKRNMEFLDLIQEGSLGLERGVEKFDPTKGYKFSTYAYWWIRQAITRAIAQQGRTIRLPIHITEKLNKIKKTQRELSQQLGRSATPAEVAKALEIDPSQIREYLSLSRQPISLDVRVGDNQDTELSELLEDEGVSPDAYITQESMRQDLQNLLAELTPQQQAVLTMRFGLNDGQELSLAKIGQHLNISRERVRQLENQALAQLKRRRANMAEYIIAS

SEQ ID NO: 35 - DNA, 1884 bp, Synechocystis sp. strain PCC6803
  CDS (1)..(1884)
atgaaattttcctggagaactgccctactttggtccctacccctgttggtagtcggctttttcttctggcaggggagctttggaggggcagatgccaacctcggttccaacactgccaacacccgcatgacctatggtcgcttcctcgaatatgtggatgctggccgcatcaccagtgtggatttatatgaaaatggccgcacggcgatcgtgcaagttagcgacccagaagtagaccggaccctccgttcccgggttgacctccccaccaatgccccggaattgattgcccgtttacgggactccaacattcgccttgattcccaccctgtccgcaacaatggcatggtttggggttttgtgggcaacttgattttccccgtgcttttgattgcttccctcttttttctcttccgccgttccagcaacatgcctgggggccccggccaagccatgaactttggtaaatccaaagctcgcttccaaatggatgccaaaaccggtgtcatgttcgatgatgtggccggtattgacgaagccaaggaagaattgcaagaggtggtaactttccttaaacagcccgaacgctttactgcagtgggggccaagattcccaaaggagtactcttagtgggccctcccggtaccggtaaaactctcctcgccaaggcgatcgccggggaagccggagttcctttcttcagcatttctggttccgagttcgtagaaatgtttgtcggcgttggtgcctcccgggtgcgggacttgtttaaaaaagccaaagagaatgccccctgtttgatcttcattgatgagattgatgccgtgggtcgtcaacggggtgctggtatcggtggtggtaacgatgaacgggaacaaaccctcaaccagctactaaccgagatggacggttttgaaggcaatacgggcattattatcattgccgccactaaccgccctgacgtgctagattctgccttgatgcgtcccggtcgtttcgatcgccaagtgatggtggatgcccctgactactctggtcgtaaggaaattttagaagtccacgcccgcaataaaaagttagcaccggaagtttccatcgactccattgcccgccgtactcccggttttagtggggctgacttggccaatttattgaatgaagccgccattctcaccgcccgccgtcgtaaatccgctatcactctgttggaaattgatgatgccgtggaccgggtggtagctggtatggaaggcacccccttggtggacagcaaaagtaagcggctaattgcttatcacgaagtaggccacgccattgtgggcacattgttaaaagaccatgatcccgtgcaaaaagtcacccttattcctcggggccaagcccaaggtttgacctggttcactcccaacgaagaacagggtttaaccaccaaagcccaactgatggcccgtattgctggagcaatgggcggtcgagccgctgaagaggaagtttttggcgatgacgaagtaaccactggggctggtggtgacctacaacaggtaactgagatggctcgccagatggtaactcgttttggcatgagcaaccttggtcccatttccctggagagttcaggtggggaagtattcctgggtggtggcttgatgaaccgttctgaatactccgaagaagtagccacccgcattgatgcccaagtacggcaattggctgaacagggtcaccaaatggctcgcaaaatcgtccaagaacaacgggaagtggttgatcgcctggtggatcttttaattgagaaagaaaccattgatggggaagaatttcggcaaattgtggcggaatacgccgaggttcccgtcaaggaacagttaattccccaactataa

SEQ ID NO: 36 - protein, 627 aa, Synechocystis sp. strain PCC6803
MKFSWRTALLWSLPLLVVGFFFWQGSFGGADANLGSNTANTRMTYGRFLEYVDAGRITSVDLYENGRTAIVQVSDPEVDRTLRSRVDLPTNAPELIARLRDSNIRLDSHPVRNNGMVWGFVGNLIFPVLLIASLFFLFRRSSNMPGGPGQAMNFGKSKARFQMDAKTGVMFDDVAGIDEAKEELQEVVTFLKQPERFTAVGAKIPKGVLLVGPPGTGKTLLAKAIAGEAGVPFFSISGSEFVEMFVGVGASRVRDLFKKAKENAPCLIFIDEIDAVGRQRGAGIGGGNDEREQTLNQLLTEMDGFEGNTGIIIIAATNRPDVLDSALMRPGRFDRQVMVDAPDYSGRKEILEVHARNKKLAPEVSIDSIARRTPGFSGADLANLLNEAAILTARRRKSAITLLEIDDAVDRVVAGMEGTPLVDSKSKRLIAYHEVGHAIVGTLLKDHDPVQKVTLIPRGQAQGLTWFTPNEEQGLTTKAQLMARIAGAMGGRAAEEEVFGDDEVTTGAGGDLQQVTEMARQMVTRFGMSNLGPISLESSGGEVFLGGGLMNRSEYSEEVATRIDAQVRQLAEQGHQMARKIVQEQREVVDRLVDLLIEKETIDGEEFRQIVAEYAEVPVKEQLIPQL

SEQ ID NO: 37 - DNA, 2619 bp, Synechocystis sp. strain PCC6803
  CDS (1)..(2619)
atgcaacccacagatcctaataaatttacggagaaagcttgggaggcgatcgccaaaacaccggagattgctaaacagcatcgacaacagcaaattgagacggaacacctactcagtgccctactagaacaaaatggtctggccaccagcatctttaataaggctggggcgagcattccccgagttaacgatcaagttaatagctttattgcccaacagccaaaattaagtaatccgagtgaatcgatttatttaggccgcagtctcgataaattgttggacaatgcggaaatagccaagtctaaatatggagacgactatatttccatcgagcacttgatggcggcttacggccaagatgaccgcctgggcaaaaacttatatcgagaaattggcctaacagaaaataagttggcagaaattatcaagcaaattagaggaacccaaaaagtgaccgatcaaaatccagagggcaaatacgaatcccttgaaaaatatgggcgagatttaacggaattagcccgggaaggtaaactagatcctgtcattggccgggatgaagaagtgcggcgcaccattcagatcctttcccgccgcacaaaaaataaccctgtgttaattggggaaccgggggttggtaaaacggcgatcgccgaaggtttagcccaaagaattattaaccatgacgtaccggaatcattgcgggatcgcaaactaatttccctcgatatgggggcgttaattgccggggcaaaataccggggggaatttgaagaaagacttaaagcggtacttaaagaagttaccgacagccaggggcaaattattctctttattgacgaaattcataccgttgtcggcgctggggccacccaaggagccatggatgcgggcaacttattgaaacccatgttagcccggggtgctttgcgttgtatcggggccaccactttagatgaatatcgcaaatatatcgaaaaagatgcggctttggaacgacgtttccaggaagttttagtggatgaacccaatgtattagataccatttccattctccggggattaaaagaacgctatgaagtacaccacggcgtaaaaattgccgatagtgccctggtagcggcggccatgttgtccaatcgttacatcagtgatcgttttctgccggataaagctattgatttagtagacgaagcagcggccaaattaaaaatggaaatcacctccaaaccagaggaattagatgaagttgaccggaaaattctccaactagaaatggagcgtttatctttacaacgggaaaatgattctgcttccaaggagcggctagaaaaattggagaaagagttggctgattttaaagaagaacagtctaaacttaatggccaatggcagtcggaaaaaacggttattgatcaaattcgtactgttaaggaaaccatcgaccaggtgaacctagaaattcaacaggcccaacgggattacgactacaataaagcagcggagttacagtatggcaaattaactgatttacagcggcaagtggaagctttggaaacccaattggcggagcaacaaacctctggcaaatccctcttacgggaagaagttttagagtctgacattgctgaaattatctcgaaatggaccggcattcccatcagtaaattggtggaatcggaaaaagaaaaactgctccacttggaagatgaactacacagccgagtgattggtcaggatgaagcggtaaccgccgtagccgaagccattcaacgctcccgagctggtctttccgatcctaatcgtcccaccgctagctttatttttctgggccccacaggggtcgggaaaactgagttagcgaaggctttggcgaaaaatttattcgacacggaagaagccctggtgcggattgatatgtctgaatatatggaaaaacacgctgtttcccgtttaatgggggcccctccgggctatgtgggctatgaagaagggggacaattgacggaagcaattcgccgccggccctattcggtcattctttttgacgagattgaaaaagcccatggggatgtgtttaacgtcatgctccaaatcctggatgatggccgtttaaccgatgcccaaggccatgtggtggacttcaaaaatacgattatcattatgaccagtaacctgggctcccaatacattttggatgtggcgggggatgatagtcgttatgaagaaatgcggagccgagttatggatgtaatgcgggaaaacttccgcccagaatttctcaatcgggtggatgaaacgattattttccatggcttacaaaaatccgagttacgatccattgtccaaattcaaattcagtctttggctacccgtttggaggaacaaaaattaactttgaagttaacggataaagccctagattttctggctgccgtgggctatgaccccgtttatggggcccgacctttaaaacgagccgtccaaaaatacctagaaacggcgatcgccaagggaattttacggggggattacaaacctggtgagaccattgtggtggatgaaaccgacgaacgcctcagttttaccagtttaaggggggatttagtcatcgtttag

SEQ ID NO: 38 - protein, 872 aa, Synechocystis sp. strain PCC6803
MQPTDPNKFTEKAWEAIAKTPEIAKQHRQQQIETEHLLSALLEQNGLATSIFNKAGASIPRVNDQVNSFIAQQPKLSNPSESIYLGRSLDKLLDNAEIAKSKYGDDYISIEHLMAAYGQDDRLGKNLYREIGLTENKLAEIIKQIRGTQKVTDQNPEGKYESLEKYGRDLTELAREGKLDPVIGRDEEVRRTIQILSRRTKNNPVLIGEPGVGKTAIAEGLAQRIINHDVPESLRDRKLISLDMGALIAGAKYRGEFEERLKAVLKEVTDSQGQIILFIDEIHTVVGAGATQGAMDAGNLLKPMLARGALRCIGATTLDEYRKYIEKDAALERRFQEVLVDEPNVLDTISILRGLKERYEVHHGVKIADSALVAAAMLSNRYISDRFLPDKAIDLVDEAAAKLKMEITSKPEELDEVDRKILQLEMERLSLQRENDSASKERLEKLEKELADFKEEQSKLNGQWQSEKTVIDQIRTVKETIDQVNLEIQQAQRDYDYNKAAELQYGKLTDLQRQVEALETQLAEQQTSGKSLLREEVLESDIAEIISKWTGIPISKLVESEKEKLLHLEDELHSRVIGQDEAVTAVAEAIQRSRAGLSDPNRPTASFIFLGPTGVGKTELAKALAKNLFDTEEALVRIDMSEYMEKHAVSRLMGAPPGYVGYEEGGQLTEAIRRRPYSVILFDEIEKAHGDVFNVMLQILDDGRLTDAQGHVVDFKNTIIIMTSNLGSQYILDVAGDDSRYEEMRSRVMDVMRENFRPEFLNRVDETIIFHGLQKSELRSIVQIQIQSLATRLEEQKLTLKLTDKALDFLAAVGYDPVYGARPLKRAVQKYLETAIAKGILRGDYKPGETIVVDETDERLSFTSLRGDLVIV

SEQ ID NO: 39 - DNA, 198 bp, Synechocystis sp. strain PCC6803
  CDS (1)..(198)
atgttcgcccccatcgttatcttggttcgtcaacagttaggcaaagctaagttcaatcagatccgcggtaaggcgattgccctccactgccagaccatcaccaacttttgtaaccgggtgggcatcgatgccaaacagcgccaaaatttaatccgtttagctaagtccaacggcaaaaccctcggtttattggcctaa

SEQ ID NO: 40 - protein, 65 aa, Synechocystis sp. strain PCC6803
MFAPIVILVRQQLGKAKFNQIRGKAIALHCQTITNFCNRVGIDAKQRQNLIRLAKSNGKTLGLLA

(19) United States
(12) Patent Application Publication
(10) Pub. No.: US 2002/0164706 A1
(43) Pub. Date: Nov. 7, 2002

(54) High level promoters from cyanobacteria

(76) Inventors: Lisa L. Huang, Hockessin, DE (US); Robert A. Larossa, West Chester, PA (US); Michael P. McCluskey, Bear, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/047,260

(22) Filed: Jan. 15, 2002

Related U.S. Application Data
(60) Provisional application No. 60/264,925, filed on Jan. 30, 2001.

(51) Int. Cl. C12P 21/02; C12N 1/21; C12N 15/74

(52) U.S. Cl. 435/69.1; 435/471; 435/252.3

(57) ABSTRACT

The invention relates to the field of microbiology. More specifically, methods are provided for the identification of highly expressed genes and their corresponding promoters and UV responsive genes and their corresponding promoters in cyanobacteria Synechocystis sp. PCC6803. These genes and promoters can be used to construct expression vectors in cyanobacteria, green algae or plants, for the production of biomaterials from sunlight, a renewable energy resource.

This application claims the benefit of U.S. Provisional Application No. 60/264,925, filed Jan. 30, 2001.

### FIELD OF THE INVENTION

The invention relates to the field of microbiology. More specifically, the invention relates to high-level expression promoters and UV responsive promoters in cyanobacteria Synechocystis sp. PCC6803.

### BACKGROUND OF THE INVENTION

The UV-B (290-320 nm) component of sunlight generates significant damage on biological systems ranging from bacteria to plants and humans. The main targets of UV-B irradiation are transfer RNA (tRNA), proteins, lipids, and, in particular, photosystems of photosynthetic organisms including plants, algae and cyanobacteria (Garcia-Pichel, Origins of Life and Evolution of the Biosphere 1998, 28:321-47). Photosynthetic organisms have adapted many different mechanisms to combat the damaging effect of UV-B irradiation, such as reducing photosynthesis and synthesizing UV protective molecules (Ehling-Schultz and Scherer, 1999. Eur. J. Phycol., 34:329-338). The latter may be of interest for use in protection of materials easily damaged by sunlight, or for developing sunscreens.

The mechanism by which photosynthetic organisms adapt to UV-B light is not completely understood. While several studies have examined the effect of UV and white light on cyanobacteria (Mate et al., J. Biol. Chem. 1998, 273 (28), 17439-17444; Li and Golden, Proc. Natl. Acad. Sci. USA, 1993, 90, 11678-11682; Ehling-Schultz and Scherer, Eur. J. Phycol. 1999, 34, 329-338; Gotz et al., Plant Physiol. 1999, 120 (2) 599-604; Sah et al., Biochem. Mol. Biol. Int. 1998, 44 (2) 245-57; Miroshnichenko Dolganov et al., Proc. Natl. Acad. Sci. USA, 1995, 92:636-640; and Mohamed and Jansson, Plant Mol Biol., 1989, 13:693-700), these authors focused on either the response of single genes or proteins to UV or white light, or certain specific molecules involved in photoprotection. None of these previous studies analyzed a near complete set of the open reading frames in Synechocystis for promoter strength and induction or repression by UV-B light in the 290-320 nm range. The identification of UV-B inducible genes and their promoters would be desirable for identifying UV-B protective compounds as well as for methods of regulating gene expression in cyanobacteria, green algae or plants, for the production of biomaterials from sunlight, a renewable energy resource.

The problem to be solved, therefore is to identify highly expressed genes and their corresponding strong promoters, and preferably UV-B inducible genes and their corresponding promoters.

Applicants have solved this problem by characterizing the global response and adaptation mechanism of cyanobacterium Synechocystis sp. PCC6803 to the stress of UV-B light using a novel DNA microarray that comprises a near complete set of open reading frames from this species. Therefore, Applicants' invention provides a group of highly expressed genes, as well as a group of UV-B inducible genes in cyanobacteria Synechocystis sp. PCC 6803 and a collection of useful strong promoters that can be used for gene over-expression either in minimal media, or in response to treatment with UV-B light. The present invention provides a unique approach for controlled overexpression of foreign genes in Synechocystis sp. PCC6803, as well as other cyanobacteria such as Synechococcus and like organisms.

### SUMMARY OF THE INVENTION

The present invention provides two sets of high level expression (i.e., strong) promoters from cyanobacteria Synechocystis sp. PCC6803. These promoters can be employed for engineering gene expression in Synechocystis sp. PCC6803 and constructing expression vectors for use in Synechocystis as well as other cyanobacteria, such as Synechococcus and like organisms. The first set of high-level expression promoters comprises promoters that demonstrate high level expression in log phase growth. The second set of promoters are induced by exposure to UV-B light.

The invention therefore provides a method for regulating expression of a coding region of interest in a cyanobacterium comprising:

a) providing a transformed cyanobacterium having a gene fusion comprising:

i) a promoter region from a gene selected from the group consisting of:

1) an amiC gene or an rbcX gene; and

2) a gene having a nucleotide sequence as set forth in SEQ ID NO: 5; and

ii) a coding region of interest;

wherein the promoter region is operably linked to the coding region of interest; and

b) culturing the transformed cyanobacterium of step (a), in the log phase whereby the promoter region is activated and the coding region of interest is expressed.

Additionally the invention provides method for regulating expression of a coding region of interest in a cyanobacterium comprising:

a) providing a transformed cyanobacterium having a gene fusion comprising:

i) a promoter region from a gene selected from the group consisting of:

1) an hliB gene, an hsp17 gene, a nblB gene, a rpoD gene, an hliA gene, a ftsH gene and a clpB gene; and

2) a gene having a nucleotide sequence selected from the group consisting of SEQ ID NOs:9, 11, 17, 21, 25, 27, 31, and 39; and

ii) a coding region of interest;

wherein the promoter region is operably linked to the coding region of interest; and

b) culturing the transformed cyanobacterium of step (a) in the presence of UV-B light, whereby the promoter region is activated and the coding region of interest is expressed.

Specific cyanobacterium useful in the present invention will be selected from the group consisting of Synechocystis and Synechococcus.

Specific coding regions of interest useful in the present invention will be selected from the group consisting of crtE, crtB, pds, crtD, crtL, crtZ, crtX crtO, phaC, phaE, efe, pdc, adh, genes encoding limonene synthase, pinene synthase, bornyl synthase, phellandrene synthase, cineole synthase, sabinene synthase, and taxadiene synthase

### BRIEF DESCRIPTION OF THE SEQUENCES

The invention can be more fully understood from the following detailed description and the accompanying sequence descriptions which form a part of this application.

Sequences contained herein are in conformity with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

| | Clone | SEQ ID | SEQ ID |
|---|---|---|---|
| Description | Name | Nucleic acid | Peptide |
| Nucleotide sequence of an | slr0447 | 1 | 2 |
| amiC gene | | | |
| Nucleotide sequence of an | slr0011 | 3 | 4 |
| rbcX gene | | | |
| Nucleotide sequence of a | sll1786 | 5 | 6 |
| gene of unknown function | | | |
| induced in log phase | | | |
| Nucleotide sequence of an | ssr2595 | 7 | 8 |
| hliB gene | | | |
| Nucleotide sequence of a | slr1544 | 9 | 10 |
| gene of unknown function | | | |
| induced by UV-B | | | |
| Nucleotide sequence of a | ss0528 | 11 | 12 |
| gene of unknown function | | | |
| induced by UV-B | | | |
| Nucleotide sequence of an | ssl1514 | 13 | 14 |
| hsp17 gene | | | |
| Nucleotide sequence of an | slr1687 | 15 | 16 |
| nblB gene | | | |
| Nucleotide sequence of a | sll1483 | 17 | 18 |
| gene of unknown function | | | |
| induced by UV-B | | | |
| Nucleotide sequence of an | sll2012 | 19 | 20 |
| rpoD gene | | | |
| Nucleotide sequence of a | ssl1633 | 21 | 22 |
| gene of unknown function | | | |
| induced by UV-B | | | |
| Nucleotide sequence of an | ssl2542 | 23 | 24 |
| hliA gene | | | |
| Nucleotide sequence of a | sll0846 | 25 | 26 |
| gene of unknown function | | | |
| induced by UV-B | | | |
| Nucleotide sequence of a | slr1674 | 27 | 28 |
| gene of unknown function | | | |
| Nucleotide sequence of an | slr1604 | 29 | 30 |
| ftsH gene | | | |
| Nucleotide sequence of a | slr0320 | 31 | 32 |
| gene of unknown function | | | |
| induced by UV-B | | | |
| Nucleotide sequence of an | sll0306 | 33 | 34 |
| rpoD gene | | | |
| Nucleotide sequence of an | slr0228 | 35 | 36 |
| ftsH gene | | | |
| Nucleotide sequence of a | slr1641 | 37 | 38 |
| clpB gene | | | |
| Nucleotide sequence of a | ssr2016 | 39 | 40 |
| gene of unknown function | | | |
| induced by UV-B | | | |

### DETAILED DESCRIPTION OF THE INVENTION

Applicants have used a novel DNA microarray to identify the global response and adaptation of cyanobacterium Synechocystis sp. PCC6803 to UV-B light and to identify strong promoters for construction of gene expression vectors in Synechocystis sp. PCC 6803. Specifically, Applicants have identified genes which are highly expressed in log phase growth and genes whose expression is highly induced by UV-B light.

Applicants' identified genes and promoters which can be used to express coding regions of interest in cyanobacteria.

In this disclosure, a number of terms and abbreviations are used. The following definitions are provided and should be helpful in understanding the scope and practice of the present invention.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. ”Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The terms “3′ non-coding sequences” or “3′ un-translated region (UTR)” refer to DNA sequences located downstream (3′) of a coding sequence and may comprise polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

”RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell.

As used herein, the term “homologous” in all its grammatical forms and spelling variations refers to the relationship between proteins that possess a “common evolutionary origin”, including proteins from superfamilies and homologous proteins from different species (Reeck et al., 1987, Cell 50:667). Such proteins (and their encoding genes) have sequence homology, as reflected by their high degree of sequence similarity

”The term homologue” when referring to a gene will mean a gene of similar function in the same or different species which may have a high degree of nucleic acid or amino acid relatedness.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

”Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

”Regulatory region” means a nucleic acid sequence which regulates the expression of a second nucleic acid sequence. A regulatory region may include sequences which are naturally responsible for expressing a particular nucleic acid (a homologous region) or may include sequences of a different origin which are responsible for expressing different proteins or even synthetic proteins (a heterologous region). In particular, the sequences can be sequences of prokaryotic, eukaryotic, or viral genes or derived sequences which stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art. An “Inducible promoter” refers to those regulated promoters that can be turned on in one or more cell types by an external stimulus or stress, such as a chemical, or light.

”Coding sequence” “coding region” or “open reading frame” (ORF) refers to a DNA sequence that codes for a specific amino acid sequence. A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence. The term “coding region of interest” refers to a coding region expressible in a cyanobacterial host.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “gene fusion” refers to the operable linking of at least two functional nucleic acid fragments which are not normally so linked in nature. Gene fusions are often comprised of promoter or regulatory regions operably linked to coding regions of other genes. Gene fusions of the present invention will typically comprise an inducible promoter operably linked to a coding region of interest.

A “polypeptide” is a polymeric compound comprised of covalently linked amino acid residues. Amino acids have the following general structure:

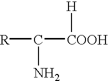

Amino acids are classified into seven groups on the basis of the side chain R: (1) aliphatic side chains, (2) side chains containing a hydroxy (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

A “heterologous protein” refers to a protein not naturally produced in the cell.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). Furthermore, the skilled artisan will recognize that the temperature and wash solution salt concentration may be adjusted as necessary according to factors such as length of the probe.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

The term “probe” refers to a single-stranded nucleic acid molecule that can base pair with a complementary single stranded target nucleic acid to form a double-stranded molecule.

As used herein, the term “oligonucleotide” refers to a nucleic acid, generally of at least 18 nucleotides, that is hybridizable to a genomic DNA molecule, a cDNA molecule, or an mRNA molecule. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid according to the invention. In another embodiment, oligonucleotides (one or both of which may be labeled) can be used as PCR primers, either for cloning full length or a fragment of a nucleic acid of the invention, or to detect the presence of nucleic acids according to the invention. In a further embodiment, an oligonucleotide of the invention can form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The term “expression”, as used herein, refers to the transcription and stable accumulation of sense (mRNA) or antisense RNA derived from the nucleic acid fragment of the invention. Expression may also refer to translation of mRNA into a polypeptide.

The term “DNA microarray” or “DNA chip” means assembling PCR products of a group of genes or all genes within a genome on a solid surface in a high density format or array. General methods for array construction and use are available (see Schena M, Shalon D, Davis R W, Brown P O., Quantitative monitoring of gene expression patterns with a complementary DNA microarray. Science. Oct. 20, 1995; 270(5235): 467-70. A DNA microarray allows the analysis of gene expression patterns or profile of many genes to be performed simultaneously by hybridizing the DNA microarray comprising these genes or PCR products of these genes with cDNA probes prepared from the sample to be analyzed. DNA microarray or “chip” technology permits examination of gene expression on a genomic scale, allowing transcription levels of many genes to be measured simultaneously. Briefly, DNA microarray or chip technology comprises arraying microscopic amounts of DNA complementary to genes of interest or open reading frames on a solid surface at defined positions. This solid surface is generally a glass slide, or a membrane (such as nylon membrane). The DNA sequences may be arrayed by spotting or by photolithography. Two separate fluorescently-labeled probe mixes prepared from the two sample(s) to be compared are hybridized to the microarray and the presence and amount of the bound probes are detected by fluorescence following laser excitation using a scanning confocal microscope and quantitated using a laser scanner and appropriate array analysis software packages. Cy3 (green) and Cy5 (red) fluorescent labels are routinely used in the art, however, other similar fluorescent labels may also be employed. To obtain and quantitate a gene expression profile or pattern between the two compared samples, the ratio between the signals in the two channels (red:green) is calculated with the relative intensity of Cy5/Cy3 probes taken as a reliable measure of the relative abundance of specific mRNAs in each sample. Materials for the construction of DNA microarrays are commercially available (Affymetrix (Santa Clara, Calif.), Sigma Chemical Company (St. Louis, Mo.), Genosys (The Woodlands, Tex.), Clontech (Palo Alto, Calif.), and Corning (Corning, N.Y.). In addition, custom DNA microarrays can be prepared by commercial vendors such as Affymetrix, Clontech, and Corning.

The term “expression profile” refers to the expression of groups of genes.

The term “gene expression profile” refers to the expression of an individual gene and of suites of individual genes.

The “comprehensive expression profile” refers to the gene expression profile of more than 75% of all genes in the genome.

A “vector” or “plasmid” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In general, a “replicon” is a unit length of DNA that replicates sequentially and which comprises an origin of replication. The term “vector” includes both viral and nonviral means for introducing the nucleic acid into a cell in vitro, ex vivo or in vivo. Viral vectors include retrovirus, adeno-associated virus, pox, baculovirus, vaccinia, herpes simplex, Epstein-Barr and adenovirus vectors. Non-viral vectors include plasmids, liposomes, electrically charged lipids (cytofectins), DNA-protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cloning vector” is a replicon, such as plasmid, phage or cosmid, to which another DNA segment may be attached so as to bring about the replication of the attached segment. Cloning vectors may be capable of replication in one cell type, and expression in another (“shuttle vector”).

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

”Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “stress”, “environmental stress”, insult” or “environmental insult” refers to any substance or environmental change that results in an alteration of normal cellular metabolism in a bacterial cell or population of cells. Environmental insults may include, but are not limited to, chemicals, environmental pollutants, heavy metals, changes in temperature, changes in pH, as well as agents producing oxidative damage, DNA damage, anaerobiosis, and changes in nitrate availability or pathogenesis.

The term “log phase”, “log phase growth”, “exponential phase” or “exponential phase growth” refers to cell cultures of organisms growing under conditions permitting the exponential multiplication of the cell number.

The term “UV-B light” means light at a wavelength of about 290 nm to about 330 nm.

The terms “UV-B light treatment”, “UV-B treatment”, “UV-B irradiation” or “UV-B exposure” mean UV-B light that is administered at an intensity of about 20 μES⁻¹ m⁻² to about 80 μES⁻¹ m⁻². Preferably, the UV-B light is administered at an intensity of about 20 μES⁻¹ m⁻².

The terms “UV-inducible” or “UV-B-inducible” gene or promoter refer to a gene or promoter whose expression or induction increases upon exposure to UV-B light.

In a specific embodiment, the term “about” or “approximately” means within 20%, preferably within 10%, and more preferably within 5% of a given value or range.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-lnterscience (1987).

DNA Microarray Analysis

The present invention provides methods for gene expression and regulation in cyanobacteria using the promoter regions from genes that are either highly expressed in log phase growth or under the influence of UV-B light. The present promoters were identified using DNA microarray technology.

It will appreciated that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Typically the genes are amplified by methods of primer directed amplification such as polymerase chain reaction (PCR) (U.S. Pat. No. 4,683,202 (1987, Mullis, et al.) and U.S. Pat. No.4,683,195 (1986, Mullis, et al.), ligase chain reaction ( LCR) (Tabor et al., Proc. Acad. Sci. U.S.A., 82, 1074-1078 (1985)) or strand displacement amplification (Walker et al., Proc. Natl. Acad. Sci. U.S.A., 89, 392, (1992) for example.

The micro-array is comprehensive in that it incorporates at least 75% of all ORF's present in the genome. Amplified ORF's are then spotted on slides comprised of glass or some other solid substrate by methods well known in the art to form a micro-array. Methods of forming high density arrays of oligonucleotides, with a minimal number of synthetic steps are known (see for example Brown et al., U.S. Pat. No. 6,110,426). The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668 which disclose methods of forming vast arrays of peptides, oligonucleotides and other molecules using, for example, light-directed synthesis techniques. See also, Fodor et al., Science, 251, 767-77 (1991).

Bacteria typically contain from about 2000 to about 6000 ORF's per genome and the present method is suitable for genomes of this size where genomes of about 4000 ORF's are most suitable. The ORF's are arrayed in high density on at least one glass microscope slide. This is in contrast to a low density array where ORF's are arrayed on a membranous material such as nitrocellulose. The small surface area of the high density array (often less than about 10 cm², preferably less than about 5 cm² more preferably less than about 2 cm², and most preferably less than about 1.6 cm.²) permits extremely uniform hybridization conditions (temperature regulation, salt content, etc.).

Once all the genes of ORF's from the genome are amplified, isolated and arrayed, a set of probes, bearing a signal-generating label are synthesized. Probes may be randomly generated or may be synthesized based on the sequence of specific open reading frames. Probes of the present invention are typically single stranded nucleic acid sequences which are complementary to the nucleic acid sequences to be detected. Probes are “hybridizable” to the ORF's. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Signal-generating labels that may be incorporated into the probes are well known in the art. For example labels may include but are not limited to fluorescent moieties, chemiluminescent moieties, particles, enzymes, radioactive tags, or light emitting moieties or molecules, where fluorescent moieties are preferred. Most preferred are fluorescent dyes capable of attaching to nucleic acids and emitting a fluorescent signal. A variety of dyes are known in the art such as fluorescein, Texas red, and rhodamine. Preferred in the present invention are the mono reactive dyes cy3 (146368-16-3) and cy5 (146368-14-1) both available commercially (i.e., Amersham Pharmacia Biotech, Arlington Heights, Ill.). Suitable dyes are discussed in U.S. Pat. No. 5,814,454 hereby incorporated by reference.

Labels may be incorporated by any of a number of means well known to those of skill in the art. However, in a preferred embodiment, the label is simultaneously incorporated during the amplification step in the preparation of the probe nucleic acids. Thus, for example, polymerase chain reaction (PCR) with labeled primers or labeled nucleotides will provide a labeled amplification product. In a preferred embodiment, reverse transcription or replication, using a labeled nucleotide (e.g. dye-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the synthesis is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Following incorporation of the label into the probe the probes are then hybridized to the micro-array using standard conditions where hybridization results in a double stranded nucleic acid, generating a detectable signal from the label at the site of capture reagent attachment to the surface. Typically the probe and array must be mixed with each other under conditions which will permit nucleic acid hybridization. This involves contacting the probe and array in the presence of an inorganic or organic salt under the proper concentration and temperature conditions. The probe and array nucleic acids must be in contact for a long enough time that any possible hybridization between the probe and sample nucleic acid may occur. The concentration of probe or array in the mixture will determine the time necessary for hybridization to occur. The higher the probe or array concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature [Van Ness and Chen (1991) Nucl. Acids Res. 19:5143-5151]. Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt./vol. glycine. Other additives may also be included, such as volume exclusion agents which include a variety of polar water-soluble or swellable agents, such as polyethylene glycol, anionic polymers such as polyacrylate or polymethylacrylate, and anionic saccharidic polymers, such as dextran sulfate. Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)) and Maniatis, supra.

The basis of gene expression profiling via micro-array technology relies on comparing an organism under a variety of conditions that result in alteration of the genes expressed. Within the context of the present invention a single population of cells was exposed to a variety of stresses that resulted in the alteration of gene expression. Specifically, expression was monitored under the conditions of exposure to UV-B light and log phase growth. Non-stressed cells are used for generation of “control” arrays and stressed cells are used to generate an “experimental”, “stressed” or “induced” arrays. Using these methods it was determined that the genes amiC and rbcX encoding a putative periplasmic binding protein and a putative chaperone respectively, were highly induced in log phase growth. Similarly, under the stress of UV-B light it was determined that hliB, hsp17, nblB, rpoD, hliA, ftsH, and the clpB genes were highly induced.

Nucleic Acids of the Invention

Two sets of high level expression (i.e., strong) promoters from cyanobacteria Synechocystis sp. PCC6803 have been identified using the above described DNA microarray technology. One set of promoters were derived from the amiC and rbcX genes and have been shown to be highly expressed in log phase growth. The second set of promoters were induced by UV-B light and consist of the genes hliB, hsp17, nblB, rpoD, hliA, ftsH, and clpB.

The amiC gene has putatively been identified as encoding a periplasmic binding protein based on sequence comparison to similar gene in public databases. amiC has been identified in Pseudomonas as being the contoller transcription antitermination in the amidase operon (Pearl et al., EMBO J. (1994), 13(24), 5810-17 and in Synechocystis (Kaneko et al., Sequence analysis of the genome of the unicellular cyanobacterium Synechocystis sp. strain PCC6803. II. Sequence determination of the entire genome and assignment of potential protein-coding regions, DNA Res. 3 (3), 109-136 (1996).

The rbcX gene has been putatively identified as a chaperone based on sequence comparisons to publicly available databases. rbcX has been identified in Synechocystis (Kaneko et al., supra) and in filamentous cyanobacteria of the genus Anabaena (Li et al. J. Bacteriol. (1997), 179(11), 3793-3796) as well as Microcystis, Tychonema, Planktothrix and Nostoc (Rudi et al., J. Bacteriol. (1998), 180(13), 3453-3461) and is thought to encode a protein that facilitates protein folding for ribulose 1,5-bisphosphate carboxylate/oxygenase. In addition to the amiC and rbcX genes, genes of unknown function have been identified as being highly induced in log phase. The most significant gene in this category has nucleic acid and amino acid sequences as set for in SEQ ID NOs:5 and 6 respectively.

Although both amiC and rbcX are known, it was not until Applicant's invention that is was appreciated that these genes were induced at high levels in the log phase and offer the promise of high level gene expression for gene fusions in cyanobacteria.

hliB and hliC have been identified as genes inducible by high light in Synechocystis (Kaneko et al., supra) and homologs have been found in higher plants and red algae (Jansson et al., Plant Molecular Biology, (January, 2000) Vol. 42, No. 2, pp. 345-351). It is thought that the hli gene product may bind chlorophyll and form dimers in the thylakoid membrane of the photosystem II complex.

hsp17 is well known to be highly expressed in response to heat stress. hsp17 is present in Synechocystis (Kaneko et al., supra) and Synechococcus (Nishiyama et al., Plant Physiology (Rockville), (May, 1999) Vol. 120, No. 1, pp. 301-308). It has been suggested that in the cyanobacteria hsp17 may play are role in the thylakoid fluidity levels of the cell membrane (Horvath et al., Proceedings of the National Academy of Sciences of the United States of America, (Mar. 31, 1998) Vol. 95, No. 7, pp. 3513-3518).

nblB has been identified in the complete genome of Synechocystis (Kaneko et al., supra) and is thought to play a role in the degradation of the light harvesting, electron transport complex phycobilisome (Dolganov et al., Journal of Bacteriology, (January, 1999) Vol. 181, No. 2, pp. 610-617).

rpoD has been identified in the complete genome of Synechocystis (Kaneko et al., supra) and is a sigma factor of chloroplast RNA polymerase used in rhodophytes (Liu et la., Journal of Phycology, (August, 1999) Vol. 35, No. 4, pp. 778-785) and other cyanobacteria (Asayama et al., Journal of Biochemistry (Tokyo), (March, 1999) Vol.125, No. 3, pp.; Caslake et al., Microbiology (Reading), (December, 1997) Vol. 143, No. 12, pp. 3807-3818; Tanaka et al., Biosci Biotechnol Biochem, (1992) 56 (7), 1113-1117).

ftsH gene has been identified in the complete genome of Synechocystis (Kaneko et al., supra), in red algae (Itoh et al., Plant Molecular Biology, (October, 1999) Vol. 41, No. 3, pp. 321-337) in E. coli., (Jayasekera et al., Archives of Biochemistry and Biophysics, (Aug. 1, 2000) Vol. 380, No. 1) and in higher plants such as tobacco (Seo et al., Plant Cell, (June, 2000) Vol. 12, No. 6, pp. 917-932). The gene product of ftsH is a metalloprotease bound to the thylakoid membrane, and degrades unassembled proteins and is involved in the degradation of the D1 protein.(Adam, Z., Biochimie (Paris), (June July, 2000) Vol. 82, No. 6-7, pp. 647-654).

The clpB gene has been identified in the complete genome of Synechocystis (Kaneko et al., supra) and in other cyanobacteria and is thought to play a role in acquired thermotolerance (Keeler et al., Plant Physiology (Rockville), (July, 2000) Vol. 123, No. 3, pp. 1121-1132).

In addition to the above mentioned UV-B inducible genes, genes of unknown function have been identified as being highly induced by UV-B light. The most significant genes in this category have nucleic acid and amino acid sequences as set for in SEQ ID NOs:9 and 10, 11 and 12, 17 and 18, 21 and 22, 25 and 26, 31 and 32 and 39 and 40, respectively.

These genes, although known in a variety of cyanobacteria and higher plants, are responsive to a diverse array of induction triggers. However, until Applicant's invention it was not appreciated that all such genes may be highly induced when the host cell is exposed to UV-B light. It will be appreciated that although these observations were made with genes isolated from the cyanobacteria Synechocystis sp. PCC6803, it will be expected that homologues of these genes in similar organisms, including higher plants will behave in a similar fashion. Homologues of these genes are those genes having similar function in related organisms and may have significant nucleotide or amino acid sequence homology over some or all of the sequence. Homologues having significant sequence homology may be identified by means well known in the art. Examples of sequence-dependent protocols for homologue identification include, but are not limited to, methods of nucleic acid hybridization, and methods of DNA and RNA amplification as exemplified by various uses of nucleic acid amplification technologies [e.g. polymerase chain reaction, Mullis et al., U.S. Pat. No. 4,683,202; ligase chain reaction (LCR), Tabor, S. et al., Proc. Acad. Sci. USA 82, 1074, (1985)] or strand displacement amplification [SDA, Walker, et al., Proc. Natl. Acad. Sci. U.S.A., 89, 392, (1992)].

Generally two short segments of the instant sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes.

Alternatively the instant sequences may be employed as hybridization reagents for the identification of homologues. The basic components of a nucleic acid hybridization test include a probe, a sample suspected of containing the gene or gene fragment of interest, and a specific hybridization method. Probes of the present invention are typically single stranded nucleic acid sequences which are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base. Hybridization methods are well defined and have been described above.

Coding region of Interest

In a specific embodiment of Applicants' invention, the coding region of interest may be either endogenous or heterologous to the cyanobacterium host cell. Any coding region that may be fused to the promoter regions of the invention and which will be expressed in a cyanobacterial host are suitable. Coding regions derived from genes that have commercial significance are preferred. A particularly preferred, but non-limiting list include, genes encoding enzymes involved in the production of isoprenoid molecules, genes encoding polyhydroxyalkanoic acid (PHA) synthases (phaE; GenBank®Accession No. GI 1652508, phaC; GenBank®Accession No. GI 1652509) from Synechocystis or other bacteria, genes encoding carotenoid pathway genes such as phytoene synthase (crtB; GenBank®Accession No. GI 1652930), phytoene desaturase (crtD; GenBank®Accession No. GI 1652929), beta-carotene ketolase (crtO; GenBank®Accession No. GI 1001724); and the like, ethylene forming enzyme (efe) for ethylene production, pyruvate decarboxylase (pdc), alcohol dehydrogenase (adh), cyclic terpenoid synthases (i.e. limonene synthase, pinene synthase, bornyl synthase, phellandrene synthase, cineole synthase, and sabinene synthase) for the production of terpenoids, and taxadiene synthase for the production of taxol, and the like. Genes encoding enzymes involved in the production of isoprenoid molecules include for example, geranylgeranyl pyrophosphate synthase (crtE; GenBank® Accession No. GI 1651762), solanesyl diphosphate synthase (sds; GenBank® Accession No. GI 1651651), which can be expressed in Synechocystis to exploit the high flux for the isoprenoid pathway in this organism. Genes encoding polyhydroxyalkanoic acid (PHA) synthases (phaE, phaC) may be used for the production of biodegradable plastics.

Microbial Expression

Once a coding region of interest has been identified a fusion with the appropriate inducible promoter region may be constructed by means well known in the art. Gene expression protocols are similar in Synechocystis and other bacteria (Maniatis, et al. supra; Donald A Bryant, The Molecular Biology of Cyanobacteria, Kluwer Academic Publisher, 1994), except the growth requirements are different (see Rippka et al., 1979, supra). Typically synechocystis is grown in BG11 media (Sigma) containing 5 mM glucose, at 30° C. illuminated with 15-50 μES⁻¹ m⁻² white light. The synechocystis cell culture is grown to mid logarithmic state, before an inducer (such as UV-B, or isopropyl thio-β-galactopyranoside) is added to induce protein expression.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. There are two kinds of preferred vectors for use in Synechocystis: self-replicating plasmids and chromosome integration plasmids. The self-replicating plasmids have the advantage of having multiple copies of coding regions of interest, and therefore the expression level can be very high. Chromosome integration plasmids are integrated into the genome by recombination. They have the advantage of being stable, but they may suffer from a lower level of expression. A specific embodiment of the present invention provides that the genetic construct resides on a plasmid in the transformed cyanobacterium. Alternatively, the genetic construct may be chromosomally integrated in the cyanobacterium genome.

Termination control regions may also be derived from various genes native to the preferred hosts. Optionally, a termination site may be unnecessary, however, it is most preferred if included.

Suitable host cells for use with the methods and promoters of the invention will include genera in the cyanobacterial family. Preferred host will include, but are not limited to the genera Asterocapsa Aphanizomenon Microcystis Cylindrospermum Anacystis, Psychrophilic Anabaena Nostoc, Tychonema, Planktothrix Lyngbya Schizothrix Nodularia Synechocystis and Synechococcus where the genera Synechocystis and Synechococcus are most preferred.

Synechocystis sp. PCC6803, a naturally competent host for transformation. DNA is directly added to actively growing cells, and plated on a selective media with the appropriate antibiotic marker. Expression of desired gene products involves growing the transformed host cells in illumination of 15-50 μEs⁻¹ m⁻² intensity of white light at 30° C., inducing expression of the transformed gene with an inducing agent, e.g., UV-B light or a chemical inducer, until cells reach a high density, e.g., optical density (OD)₇₃₀ₙₘ=4. Cells are harvested and gene products are isolated according to protocols specific for the gene product. Other host cells may also be used within the scope of the invention, including but not limited to other species of Synechocystis, Synechococcus species, other cyanobacteria, and the like.

Culture Conditions

Once a gene fusion comprising an inducible promoter region operably linked to a coding region of interest is inserted into an appropriate host cell, the expression of the coding region may be controlled by regulating the inducer. In the case of a fusion comprising the amiC or rbcX gene the cells need only be grown in the log phase for induction and expression to occur. Where the fusion comprises any of the UV-B light inducible promoter regions, the cultures must be exposed to a suitable UV-B wavelength and at a suitable intensity. Wavelengths of about 290 nm to about 330 nm are preferred and a light intensity of about 20 μES⁻¹ m⁻² to about 80 μES⁻¹ m⁻² is suitable

Where commercial production of a protein encoded by a gene fusion is desired a variety of culture methodologies may be applied. For example, large scale production of a specific gene product, overexpressed from a recombinant microbial host may be produced by both Batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992), herein incorporated by reference.

Alternatively, commercial production of proteins encoded by the instant gene fusions may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

General Methods

Standard recombinant DNA and molecular cloning techniques used herein are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Synechocystis sp. PCC6803 used in the following examples is available from the American Type Culture Collection, accession number ATCC 27184 (Ripka et al., 1979. J. Gen. Micro., 111:1-61).

Synechocystis sp. PCC6803 DNA Microarray Preparation

Synechocystis DNA microarray slides were prepared using a Molecular Dynamics Genll Spotter (Molecular Dynamics, Sunnyvale Calif.). A collection of purified PCR products of all Synechocystis open reading frames were transferred from 384 well microtiter plates to microarray glass slides using the GenIII spotter. The spotted slides were stored in desiccated container at room temperature where they were stable for about three months.

Hybridization of Microarray Slides and Quantitation of Gene Expression

Microarray glass slides (Molecular Dynamics, Sunnyvale Calif.) were treated with 100% isopropanol for 10 min, boiling double distilled water for 5 min, then treated with blocking buffer (3.5×SSC, 0.2% SDS, 1% BSA) for 20 min at 60° C., rinsed five times with double distilled water, then twice with isopropanol, followed by drying under nitrogen. Typically 100 picomoles of Cy3 labeled cDNA probes were prepared from total RNA isolated from the UV-B treated Synechocystis culture and mixed with an equal amount of Cy5 labeled cDNA probes prepared from total RNA isolated from the untreated Synechocystis culture. These were applied to a glass slide in a total volume of 30 μL. The hybridization was repeated using 100 picomoles of Cy5 labeled cDNA probes prepared from total RNA isolated from UV-B treated Synechocystis culture mixed with an equal amount of Cy3 labeled cDNA probes prepared from total RNA isolated from the untreated culture. These were applied to a second glass slide in a total volume of 30 μL. The hybridization reactions on the glass slides were incubated for 16 hr at 42° C., in a humidified chamber. Hybridized slides were washed in 1×SSC (0.15 M NaCl, 0.015 M sodium citrate), 0.1% SDS for 5 min at42° C.; 0.1×SSC, 0.1% SDS for 5 min at 42° C.; three washes in 0.1×SSC for 2 min at room temperature; rinsed with double distilled water and then with 100% isopropanol; and dried under nitrogen. The slides were scanned using a Molecular Dynamics laser scanner for imaging of Cy3 and Cy5 labeled cDNA probes. The images were analyzed using Array Vision Software (Molecular Dynamics, Sunnyvale, Calif.) to obtain fluorescence signal intensities of each spot (each ORF on the array) to quantitate gene expression. The normalized ratio between the signals in the two channels (red:green) is calculated and the relative intensity of Cy5/Cy3 probes for each spot represents the relative abundance of specific mRNAs in each sample

Minimal media was used in many of the cultures of the following examples and means a growth media composed of various salts required for the growth of the microbial/bacterial strain. In general, minimal media lacks amino acids, peptides, and sugars, and is commercially available from GIBCO (Grand Rapids Mich.).

The meaning of abbreviations is as follows: “h” and “hr” mean hour(s), “min” means minute(s), “sec” or “s” mean second(s), “d” means day(s), “mL” means milliliters, “L” means liters, “μg” means micrograms, “mg” means milligrams, “pmol” means pico moles, “μM” means micromolar, “mM” means millimolar, “M” means molar, “nm” means nanometer(s), “m” means meter(s), “OD” means optical density, “rpm” means revolutions per minute, and “μE” means microeinstein(s), wherein 1 μE equals 10₋₆ moles of photons.

### Example 1

### Preparation of Synechocystis sp. PCC6803 cDNA Probes

Example 1 describes the construction of Synechocystis sp. PCC6803 cDNA probes following growth of the cells in either minimal growth media (control) or minimal media plus UV-B light treatment. The cDNA probes were used to determine gene expression patterns of many genes simultaneously on a Synechocystis sp. PCC6803 DNA microarray as described in Examples 2 and 3 below.

Synechocvstis Strain and Culture Methods

Briefly, Synechocystis sp. PCC6803 cells were grown at 30 μES⁻¹ m⁻² light intensity in a minimal growth media, BG-11 (Rippka, R., Deruelles, J., Waterbury, J. B., Herdman, M., Stanier, R. Y. (1979) J. Ben. Microbiol. 111, 1-61)) at 30° C., with shaking at 100 rpm. Fifty milliliters of Synechocystis cells grown to mid logarithmic phase (OD₇₃₀ₙₘ=0.8 to 1.0) were divided into two 25 mL cultures and transferred from the Erlenmeyer growth flask to two 100 mL plastic Petri dishes. The Petri dishes were placed on a rotary shaker and shaken at 100 rpm.

Cell Treatments

For the control, the Petri dishes comprising the Synechocystis cells were placed on a rotary shaker with the lids on, and shaken at 100 rpm for 20 min or 2 hr. For the UV-B treated group, the Petri dishes comprising the Synechocystis cells were placed on a rotary shaker with the lids on, and shaken at 100 rpm for 20 min or 2 hr. A UV-B lamp (UVM-28, mid range at 302 nm, Ultra Violet Products, Upland, Calif.) was positioned above the Petri dishes and the distance between the UV-B light source and the Petri dishes was adjusted to give the desired level of UV-B light intensity. The level of UV-B light intensity was measured at the surface of the cell culture using UVX-31 radiometer (Ultra-Violet Products, Upland, Calif.), following the manufacturer's instructions. UV-B treatment was performed with the lid on for either 20 min or 120 min. Following UV-B irradiation, the cells were immediately cooled on ice and their RNA isolated as described below.

Total RNA Isolation and cDNA Probe Synthesis

Control Synechocystis cells and UV-B treated Synechocystis cells were cooled rapidly on ice and centrifuged at 3200×g for 5 min. Total RNA samples were isolated using Qiagen RNeasy® Mini Kit (Qiagen, Valencia, Calif.), following the manufacturer's protocol. RNase A digestion was performed according to the manufacturers instructions, and a second round of purification was performed using the RNeasy® Mini Kit. The purified total RNA was analyzed by agarose gel electrophoresis.

From each total RNA preparation, both Cy3 and Cy5 florescent dye labeled cDNA probes were prepared. To synthesize the Cy3 or Cy5 labeled cDNA probes, a reverse transcription reaction was performed using 10 μg total RNA, 12 μg random hexamer (Ambion, Austin, Tex.), 50 μM of dATP, dGTP, dTTP, 25 μM of dCTP, and 15 μM Cy3-dCTP or 22 μM Cy5-dCTP (Amersham Pharmacia Biotech, Piscataway N.J.), 10 mM DTT, 50 mM Tris-HCl pH 8.3, 75 mM KCl, 15 mM MgCl₂ and 4 units of AMV reverse transcriptase (Gibco BRL-Life Technologies, Rockville, Md.)) in total volume of 40 μL. The reaction was carried out at 42° C. for 2.5 hr. After the labeling reaction, RNA templates were degraded by alkaline hydrolysis and the cDNA probes were purified using Qiagen PCR purification kit. The purified probes were quantitated by measuring the absorbance at 260 nm, 550 nm (Cy5 dye incorporation) and 650 nm (Cy3 dye incorporation). Prior to hybridization, 100-200 pmol of the purified Cy3 or Cy5 labeled cDNA probes were dried under vacuum, and re-dissolved in the hybridization buffer (5×SSC, 50% formamide, 0.1% SDS, and 0.03 mg/mL salmon sperm DNA).

### Example 2

### Analysis of Synechocystis sp. PCC6803 Gene Expression in Minimal Media

Example 2 describes the identification of the most highly expressed genes and their corresponding strong promoters in Synechocystis sp. PCC6803 when grown in log phase in BG11 media containing 5 mM glucose as described above.

Specifically, a DNA microarray was prepared according to the methods described above using PCR amplified open reading frames and using genomic Synechocystis sp. PCC6803 DNA as template. Synechocystis sp. PCC6803 gene expression was determined by hybridizing this DNA microarray as described above with fluorescent cDNA probes synthesized from total RNA isolated from Synechocystis sp. PCC6803 cells grown in BG11 media containing 5 mM glucose as described in Example 1.

Briefly, for each duplicated minimal media experiment, two hybridization reactions were performed as described above. Specifically, the first reaction used equal molar (typically 100-200 pmol incorporated florescent dye) of Cy5-labeled cDNA from total RNA of the minimal media grown sample, and Cy3-labeled cDNA probes from the same sample. The second reaction used both Cy5 and Cy3-labeled cDNA synthesized from Synechocystis sp. PCC6803 genomic DNA. The signal intensities were quantitated as described above. To calculate the relative expression level of each Synechocystis gene in cells grown in the minimal media, the average normalized signal intensity of the hybridized cDNA probes was divided by the average signal intensity of the hybridized cDNA probes from genomic DNA. Analysis of the data from these microarray experiments indicated that the most highly expressed genes, i.e., those genes that are under the control of the strongest promoters, in Synechocystis grown in log phase under these minimal media conditions (see Table 1).

TABLE 1
Most highly expressed genes in Synechocystis sp. PCC6803
in minimal growth media (BG11 + 5 mM glucose).

| | | | Transcript | | |
|---|---|---|---|---|---|
| | | | copy in total | | |
| | | | mRNA | | |
| Systematic | | | (Average | SEQ ID NO: | |
| Name | Gene | Function | copy = 1) | NA** | AA*** |
| slr2051 | cpcG | Phycobilisome rod-core linker | 64.91 | | |
| | | polypeptide CpcG | | | |
| sll1580 | cpcC | Phycocyanin associated linker protein | 22.71 | | |
| slr0447 | amiC | Putative periplasmic binding protein | 19.45 | 1 | 2 |
| sll1070 | tktA | Transketolase | 19.24 | | |
| sll0018 | cbbA | Fructose-1, 6-bisphosphate aldolase | 14.27 | | |
| slr0011 | rbcX | Putative chaperone | 12.00 | 3 | 4 |
| ssl0563 | psaC | photosystem I subunit VII | 11.31 | | |
| slr1655 | psaL | photosystem I subunit XI | 10.91 | | |
| sll0819 | psaF | photosystem I subunit III | 10.56 | | |
| sll1867 | psbA3 | photosystem II D1 protein | 10.43 | | |
| sll1324 | atpF | ATP synthase subunit b | 10.37 | | |
| sll1746 | rpl12 | 50S ribosomal protein L12 | 10.13 | | |
| sll1099 | tufA | protein synthesis elongation factor Tu | 9.48 | | |
| slr0009 | rbcL | ribulose bisphosphate carboxylase large | 8.39 | | |
| | | subunit | | | |
| slr0012 | rbcS | ribulose bisphosphate carboxylase small | 8.14 | | |
| | | subunit | | | |
| sll1326 | atpA | ATP synthase a subunit | 7.72 | | |
| slr1908 | | ND* | 7.62 | | |
| sll1578 | cpcA | phycocyanin a subunit | 7.60 | | |
| slr2067 | apcA | allophycocyanin a chain | 7.51 | | |
| slr2052 | | ND* | 7.41 | | |
| sll1184 | ho | heme oxygenase | 7.27 | | |
| ssl3437 | rps17 | 30S ribosomal protein S17 | 7.26 | | |
| sll1786 | | hypothetical protein (ND*) | 7.16 | 5 | 6 |
| ssl0020 | petF | ferredoxin | 7.07 | | |
| sll1812 | rps5 | 30S ribosomal protein S5 | 7.04 | | |

*ND = not determined
**NA = nucleic acid SEQ ID NO.
***AA = amino acid SEQ ID NO.

### Example 3

### Analysis of Synechocystis sp. PCC6803 Gene Expression Following UV-B Exposure

Example 3 describes the identification of the most highly UV-B responsive genes in Synechocystis sp. PCC6803 when grown under minimal media conditions and exposed to 20 minutes of UV-B irradiation at 20 μES⁻¹ m⁻² intensity. These UV inducible promoters can be used to control expression of certain proteins that may be toxic to Synechocystis cells. Microarrays and probes were prepared for UV-B induced and non-induced experiments essentially as described above using Synechocystis sp. PCC6803.

Specifically, a DNA microarray was prepared according to the methods described above using DNA isolated from Synechocystis sp. PCC6803. For each UV-B treatment experiment, two hybridization reactions were performed. In particular, the first reaction used equal molar (typically 100-200 pmol) Cy5-labeled cDNA made from total RNA isolated from the UV-B treated sample, and Cy3-labeled cDNA from total RNA isolated from the control sample (Synechocystis sp. PCC6803 grown in BG11 media containing 5 mM glucose). The second reaction used Cy3-labeled cDNA made from total RNA isolated from the UV-B treated sample, and Cy5-labeled cDNA made from total RNA isolated from the control sample. The signal intensities were quantitated as described above. To calculate the ratio of fold induction (i.e., UV-B/control), the UV-B treated sample signal intensities were divided by the signal intensities of the control sample. Since there were two sets of data from duplicate spotting within each slide, the total number of gene expression measurements for each gene was four. All four induction ratios for each gene were analyzed to determine the standard deviation, an indicator of the level of confidence for the specific data set for each gene.

Analysis of the date defined the most highly UV-B induced genes in Synechocystis following UV-B treatment (see Table 2). Only genes whose expression was induced more than 4 fold by UV-B light (20 min at 20 μES⁻¹ m⁻² intensity) as compared to the minimal media control are listed in Table 2.

In addition to genes of known function in the group of UV inducible genes, there are several genes of unknown function: slr1544, sll0528, ssll0846, slr1674, slr0320, and sr2016. The results tabulated in table 2 is the first level of functional assignment for these genes. The promoters of these genes can be used to construct UV inducible expression vectors in Synechocystis.

TABLE 2
Most highly induced genes in Synechocystis sp. P006803 in BG11 media
containing 5 mM glucose, with 20 min of UV-B treatment at 20 μES⁻¹ m⁻² intensity

| Systematic | | | Data/ | | SEQ ID NO: | |
|---|---|---|---|---|---|---|
| Name | Gene | Function | Control | STD | NA** | AA*** |
| ssr2595 | hliB | High light-inducible protein | 22.7 | 4.7 | 7 | 8 |
| slr544 | | ND* | 15.5 | 7.6 | 9 | 10 |
| sll0528 | | ND* | 12.1 | 3.9 | 11 | 12 |
| sll1514 | hsp17 | small heat shock protein | 9.9 | 3.9 | 13 | 14 |
| slr1687 | nblB | phycobilisome degradation protein NblB | 8.2 | 1.9 | 15 | 16 |
| sll1483 | | transforming growth factor induced protein | 7.8 | 2.2 | 17 | 18 |
| sll2012 | rpoD | RNA polymerase sigma factor | 6.3 | 2.0 | 19 | 20 |
| ssl1633 | | CAB/ELIP/HLIP superfamily | 6.0 | 1.0 | 21 | 22 |
| ssl2542 | hliA | high light-inducible protein | 5.6 | 1.6 | 23 | 24 |
| sll0846 | | ND* | 4.7 | 0.9 | 25 | 26 |
| slr1674 | | ND* | 4.7 | 1.8 | 27 | 28 |
| slr1604 | ftsH | Chloroplast associated protease FtsH | 4.6 | 1.9 | 29 | 30 |
| slr0320 | | ND* | 4.5 | 2.2 | 31 | 32 |
| sll0306 | rpoD | RNA polymerase sigma factor | 4.4 | 1.0 | 33 | 34 |
| slr0228 | ftsH | cell division protein FtsH | 4.3 | 1.7 | 35 | 36 |
| slr1641 | clpB | ClpB protein | 4.3 | 1.1 | 37 | 38 |
| ssr2016 | | ND* | 4.2 | 2.2 | 39 | 40 |
| sll1867 | psbA3 | photosystem II D1 protein | 4.1 | 0.3 | | |

*ND = not determined
**NA = nucleic acid SEQ ID NO.
***NA = amino acid SEQ ID NO.

What is claimed is:

1. A method for regulating expression of a coding region of interest in a cyanobacterium comprising:
a) providing a transformed cyanobacterium having a gene fusion comprising:
i) a promoter region from a gene selected from the group consisting of:
1) an amiC gene or an rbcX gene; and
2) a gene having a nucleotide sequence as set forth in SEQ ID NO: 5; and
ii) a coding region of interest;
wherein the promoter region is operably linked to the coding region of interest; and
b) culturing the transformed cyanobacterium of step (a), in the log phase whereby the promoter region is activated and the coding region of interest is expressed.

2. A method according to claim 1, wherein the promoter region is from a gene encoding a polypeptide having the amino acid sequence selected from the group consisting of SEQ ID NO:2, SEQ ID NO:4 and SEQ ID NO:6.

3. A method for regulating expression of a coding region of interest in a cyanobacterium comprising:
a) providing a transformed cyanobacterium having a gene fusion comprising:
i) a promoter region from a gene selected from the group consisting of:
1) an hliB gene, an hsp17 gene, a nblB gene, a rpoD gene, an hliA gene, a ftsH gene and a clpB gene; and
2) a gene having a nucleotide sequence selected from the group consisting of SEQ ID NOs:9, 11, 17, 21, 25, 27, 31, and 39; and
ii) a coding region of interest;
wherein the promoter region is operably linked to the coding region of interest; and
b) culturing the transformed cyanobacterium of step (a) in the presence of UV-B light, whereby the promoter region is activated and the coding region of interest is expressed.

4. A method according to claim 3, wherein the promoter region is from a gene encoding a polypeptide having the amino acid sequence selected from the group consisting of SEQ ID NOs:8, 10, 12, 14, 16, 18, 20, 22, 24, 26, 28, 30, 32, 34, 36, 38, and 40.

5. A method according to claim 3, wherein the UV-B light has a wavelength of from about 290 nm to about 330 nm.

6. A method according to claim 3, wherein the UV-B light has an intensity of from about 20 μES⁻¹ m⁻² to about 80 μES⁻¹ m⁻².

7. A method according to either of claims 1 or 3, wherein the cyanobacterium is selected from the group consisting of Asterocapsa Aphanizomenon Microcystis Cylindrospermum Anacystis psychrophilic Anabaena Nostoc, Tychonema, Planktothrix Lyngbya Schizothrix Nodularia Synechocystis and Synechococcus.

8. A method according to claim 7, wherein the cyanobacterium is selected from the group consisting of Synechocystis and Synechococcus

9. A method according to either of claims 1 or 3, wherein the promoter region is derived from a cyanobacterium.

10. A method according to claim 9, wherein the promoter region is derived from the group consisting of Asterocapsa Aphanizomenon Microcystis Cylindrospermum Anacystis psychrophilic Anabaena Nostoc, Tychonema, Planktothrix Lyngbya Schizothrix Nodularia Synechocystis and Synechococcus.

11. A method according to claim 10, wherein the promoter region is derived from the group consisting of Synechocystis and Synechococcus.

12. A method according to either of claims 1 or 3, wherein the coding region of interest is endogenous to the cyanobacterium.

13. A method according to either of claims 1 or 3, wherein the coding region of interest is heterologous to the cyanobacterium.

14. The method according to either of claims 1 or 3, wherein the coding region of interest is selected from the group consisting of crtE, crtB, pds, crtD, crtL, crtZ, crtX crtO, phaC, phaE, efe, pdc, adh, genes encoding limonene synthase, pinene synthase, bornyl synthase, phellandrene synthase, cineole synthase, sabinene synthase, and taxadiene synthase

15. The method according to either of claims 1 or 3, wherein the gene fusion resides on a plasmid in the transformed cyanobacterium.

16. The method according to either of claims 1 or 3, wherein the gene fusion is chromosomally integrated in the cyanobacterium genome.
